(19) United States
(12) Patent Application Publication
(10) Pub. No.: US 2020/0370101 A1
(43) Pub. Date: Nov. 26, 2020

(54) APOE PROMOTOR SNP ASSOCIATED WITH RISK OF ALZHEIMER'S DISEASE AND THE USE THEREOF

(71) Applicant: IFOMEDITECH CO.,LTD, Seoul (KR)

(72) Inventors: Kun Ho Lee, Gwangju (KR); Kyu Yeong Choi, Gwangju (KR); Jang Jae Lee, Gwangju (KR)

(73) Assignee: IFOMEDITECH CO., LTD., Seoul (KR)

(21) Appl. No.: 15/932,345

(22) PCT Filed: Jan. 15, 2018
(86) PCT No.: PCT/KR2018/000649

§ 371 Date: Feb. 16, 2018

(30) Foreign Application Priority Data
May 15, 2017 (KR) 10-2017-0060225
May 17, 2017 (KR) PCT/KR2017/005137

(51) Int. Cl. C12Q 1/6827; C12Q 1/6844; C12Q 1/6883; G01N 33/68; A61B 5/00

(52) U.S. Cl. CPC C12Q 1/6827; C12Q 1/6844; C12Q 1/6883; C12Q 2600/156; A61B 5/0042; G01N 2800/2821; G01N 33/6896

(57) ABSTRACT

The present invention is about SNPs which can be used to predict a risk of AD. The polymorphism, rs405509 is located at the position of 44905579 of 19^{th} chromosome based on GRCh38.p7 version. According to the present invention, the rs405509 T allele increases the risk of AD in addition to APOE E4/E4. Furthermore, according to the analysis results for the cortical and hippocampal thickness between APOE genetic types, more severe atrophy was observed for the E4/4 subjects comparing to E3/E3 subjects in East Asian. In addition, still more severe cortical thickness atrophy was confirmed for the rs405509 T/T subjects in Caucasians having E4/E4 genotypes. Therefore, the present invention can be used to diagnose or predict a risk of AD and/or AD associated dementia, by determining genetic variations of both APOE E4/E4 and rs405509 T/T.

### TECHNICAL FIELD

The present invention relates to single nucleotide polymorphisms (SNPs) for early diagnosis and risk prediction of Alzheimer's disease. In addition, this invention relates to a method for early diagnosis and risk prediction of Alzheimer's disease by using the SNPs.

### BACKGROUND ART

Alzheimer's disease is a progressive neurodegenerative disorder, characterized by cognitive decline and senile plaques in the brain. It is the most common cause of dementia in the elderly, accounting 60˜80% of cases, and it has become a global health issue (Barnes and Yaffe, 2011). The prevalence of Alzheimer's disease was estimated at approximately 13% of elderly over the age of 65 years and 45% over the age of 85 years. Alzheimer's disease (AD) is a complicated neurodegenerative disorder, characterized by memory loss and other cognitive impairment, as well as brain structure alterations (Ballard et al., 2011; Forero et al., 2006). By neuropathological measures, AD patients show reduced hippocampal volumes, increased amyloid plaques in posterior cingulate cortex (PCC), medial prefrontal cortex, and hippocampus. In addition, the patients show reduced beta-amyloid level and increased tau and p-tau levels in CSF (Jack, 2012; Trojanowski et al., 2010).

Though agents for treating Alzheimer's-associated dementia have been developed, there is no effective agent. Therefore, early diagnosis and prediction of Alzheimer's-associated dementia are highly required. The methods for diagnosing dementia include neurophysiological tests, MRI brain imaging, clinical diagnosis by a medical doctor and pathological diagnosis by detecting amyloid-PET using cerebrospinal fluid (CSF) and florbetaben. The onset of dementia or mild cognitive impairment may be diagnosed by clinical diagnosis. However, it is sometimes not easy to distinguish these disorders from other brain disorders. Further, it is not appropriate for early diagnosis since the clinical diagnosis is possible only after progression of symptoms of the disorders. The cerebrospinal fluid analysis is reliable method for diagnosing dementia since it is performed by quantitative analysis of beta-amyloid and tau proteins. However, the invasive CSF sampling makes the subjects reluctant to this analysis. The pathological diagnosis by detecting amyloid-PET is highly reliable but expensive. In addition, the MRI brain imaging technologies have been developed to determine brain damages, such as, cortical atrophy and hippocampus, etc., which accompany to dementia. It is known, however, that the early diagnosis of dementia is not possible by MRI brain imaging technologies currently available. Further, various technologies for diagnosing dementia using blood samples are being developed, but these have limitations in the number of blood samples necessary for diagnosis and the accuracy thereof. Therefore, the verification of reliability of the technologies is still required for clinical use.

Among the genetic factors, the mutations in amyloid precursor protein, presenilin-1, and presenilin-2 are primary causal factors for early onset, familial Alzheimer's disease (Blennow et al., 2006; Hardy and Selkoe, 2002), and e4 allele in APOE gene is strong genetic risk factor for late onset, sporadic Alzheimer's disease (Bu, 2009; Corder et al., 1993; Huang and Mucke, 2012). APOE gene has three polymorphic alleles, e2, e3, and e4, of which frequencies are 8.4%, 77.9%, and 13.7% worldwide (Farrer et al., 1997). However, e4 frequency in Alzheimer's disease is dramatically increased to about 40% (Farrer et al., 1997). APOE is located on human chromosome 19q13.2, and is known to include 6 (six) genotypes polymorphisms (E2/E2, E2/E3, E3/E3, E2/E4, E3/E4, E4/E4) resulting from the combinations of 3 (three) alleles (e2, e3 and e4) which are formed by the variations of 112th (Cys/Arg) and 158^{th} (Arg/Cys) amino acids. The APOE e4 is typically present in more than 50% of AD patients, but is found only in nearly 15% of cognitively normal controls (Ward et al., 2012). Previous studies have shown that APOE e4 tends to move the age of onset 5˜15 years earlier (Corder et al., 1993; Gomez-Tortosa et al., 2007). In addition, a few studies reported the effect of APOE e4 on cognitive decline, in which some studies suggested the cognitive decline by APOE e4, some studies suggested no effect, and other studies suggested the slower cognitive decline in APOE e4 carrier (Anstey and Christensen, 2000; Beaudreau et al., 2013; Caselli et al., 2009; Craft et al., 1998; Deary et al., 2002; Jorm et al., 2007; Kleiman et al., 2006; Mayeux et al., 2001; Van Gerven et al., 2012). In spite of other controversial results, it has reported that APOE e4 healthy homozygotes showed the reduced hippocampal volumes, whereas e4 heterozygotes did not show the difference from e4 non-carriers in heathy aging group (Crivello et al., 2010; Farlow et al., 2004; Lemaitre et al., 2005). In addition, APOE e4 has been shown to be involved in conversion from healthy aging and mild cognitive impairment to Alzheimer's disease (Wang et al., 2011). The risk of e4 allele has been reported to be diverse between ethnic groups (Brainerd et al., 2013; Farrer et al., 1997; Heun et al., 2010; Hsiung and Sadovnick, 2007; Hsiung et al., 2004; Rose, 2005). However, the prediction of Alzheimer's disease based on the e4 allele can explain only less than 20% of the effect of genetic factors on dementia. In this regard, it is insufficient for clear elucidation of ethnic difference in e4-mediated risk for Alzheimer's disease and the possible causal factor in detail. Meanwhile, the Korean Patent Registration No.: 10-1335021 disclosed the relevance between patients suffering from Alzheimer's disease or mild cognitive impairment and T/G heterozygotes of APOE rs405509.

We, inventors have studied not only APOE E4 genetic mutations but also other genetic mutations, which may influence on risk for Alzheimer's disease, surrounding APOE gene. Furthermore, we have confirmed that there are ethnic differences regarding risk for dementia of the APOE E4/E4 homozygotes, and have studied to determine the reasons thereof. As a result, we have confirmed that SNPs located in APOE promoter are responsible for the ethnic difference of APOE E4/E4 in the risk for Alzheimer dementia, and the allele influences on the thickness of cerebral cortex. Further, we inventors determined the influence of APOE polymorphisms on the brain structure by analyzing the thickness of the cerebral cortex and the volume of the hippocampus as well.

### DISCLOSURE

### Technical Problem

The purpose of this invention is to provide single nucleotide polymorphisms (SNPs) for predicting Alzheimer's disease and/or the risk of dementia caused by Alzheimer's disease.

Another purpose of this invention is to provide a method for predicting Alzheimer's disease and/or the risk of dementia caused by Alzheimer's disease, by determining the said SNPs.

### Technical Solution

The present invention provides single nucleotide polymorphism (SNP) relating to Alzheimer's disease and/or the risk of dementia caused by Alzheimer's disease. More specifically, the above SNP is rs405509, which is located at 19th chromosome, 44905579, based on human genome map GRCh38.p7, and its alleles are T and G. It also shows difference in minor allele frequency (MAF) between ethnic groups.

In one example of the present invention, genomic DNAs are extracted and purified from experimental samples (for example, cells, tissues, or body fluids) isolated from subjects, and then rs405509 SNP polymorphisms and APOE genotypes of APOE promoter are analyzed and determined to predict the risk of dementia.

In another example of the present invention, a method for providing information to predict Alzheimer's dementia is provided, wherein the method employs APOE £ genetic variation (rs429358 and rs7412) and rs405509 genetic variation.

In yet another example of the present invention provides a method for diagnosing MIC (minor cognitive impairment) or Alzheimer's disease or predicting the risk of MIC or Alzheimer's disease by determining genetic variations, which indicate the MIC or AD, from a sample isolated from a subject and comprising nucleic acids, wherein the method includes steps of, (a) contacting the sample above with a reagent to confirm the presence or absence of the said genetic variations in genes encoding APOE E4 allele or genes selected from genetic products of the said genes encoding APOE E4 allele; and (b) contacting the sample above with a reagent to detect the presence or absence of the said genetic variations in genes encoding rs405509 T allele of APOE promotor or genes selected from genetic products of the said genes encoding APOE E4 allele; and wherein the presence of both genetic variations of APOE E4/E4 and rs405509 T/T indicates that the subject is a MIC or AD patient or indicates that the subject has a risk of MIC or AD disease. In this example, the said method may include amplifying nucleic acids isolated from the subject, and the nucleic acids may be genomic DNAs or RNAs.

In yet another example of the present invention, the said method may further include obtaining images of cortical thickness, wherein the cortical thickness atrophy determined by the images presents that the subject is a MIC or AD patient or presents that the subject has a risk of MIC or AD.

In yet another example of the present invention, the said method may further include performing any one or more tests selected from the group consisting of neurophysiological test, CSF analysis and Amyloid-PET test.

In one example of the present invention, the method of determining the presence or absence of genetic variations may include constructing a complex of the said nucleic acids in single-stranded form and their complementary primer sets, and analyzing the presence or absence of the said genetic variations in the said complex. The presence or absence of the said genetic mutations can be determined by a method, for example, polymerase chain reaction, nuclease digestion, hybridization, southern blotting, restriction enzyme fragment polymorphism, sequencing, primer extension, or single-stranded conformation polymorphism, or any combination of two or more of the above.

In another example of the present invention, a kit for diagnosing MIC or Alzheimer's disease or predicting the risk of MIC or Alzheimer's disease is provided. The said kit may determine the presence or absence of the said genetic variations in genes encoding APOE E4 allele or genes selected from the genetic products of the genes encoding APOE E4 allele; and further determine the presence or absence of rs405509 T/T genetic variations in genes encoding rs405509 T/T allele of APOE promotor or genes selected from the genetic products of the genes encoding rs405509 T/T allele of APOE promotor. Thus, the kit above may further include primer sets complementary to the APOE E4 allele, the APOE promotor SNPs, rs405509 T/T, T/G and GG, respectively, and enzymes for reaction.

The gene products of the present invention may include RNA, for example, tRNA, mRNA, rRNA, miRNA and siRNA, polypeptides; and proteins.

In yet another example of the present invention, the said kit may further include a manual for analyzing gene mutations or for amplifying nucleic acids; or include a standard control.

In yet another example of the present invention, the complementary primer sets for determining presence or absence of the said gene variations in the genes encoding APOE E4 allele or the genes selected from gene products of the genes encoding APOE E4 allele are provided. Further, genes encoding rs405509 T alleles of APOE promotor or gene products thereof may be additionally provided. Therefore, in one example of the present invention, the said primer sets are complementary to APOE SNP, APOE E4, and APOE promotor SNP, rs405509 T/T, T/G and GG, respectively.

In yet another example of the present invention, a label, for example, a radio-isotope, a fluorescence or a chemical derivative, may be attached to the primer sets.

### Advantageous Effects

According to the present invention, the risk of Alzheimer dementia can be diagnosed more accurately by determining APOE genotype, APOE E4/E4, as a genetic variation for predicting sporadic Alzheimer dementia and, in particular, further detecting genetic variations of rs405509.

### DESCRIPTION OF DRAWINGS

FIG. 1A is a flow diagram showing the strategy of screening the candidate polymorphisms modulating the risk of APOE e4/4. FIG. 1B shows the schematic diagram of APOE gene structure. The SNPs of rs449647 (−491 A/T), rs405509 (−219 T/G), rs440446 (+113 G/C), rs429358, and rs7412 are depicted in Panel B. FIG. 10 shows distributions of genotype frequencies for rs405509, rs449647, rs440446 were assessed for whole case-control subjects or e4 homozygotes from East Asian, Caucasian and African ancestry. The genotype frequencies were calculated as an average of frequencies in each case and control.

FIG. 2 shows cortical thinning maps in East Asians and Caucasian (FIG. 2A). General linear model was applied to infer the point-wise cortical thickness differences using APOE e4/4 and e3/3 genotypes as fixed factor and age, gender and field strengths as covariates. The figures show the statistical difference (p<0.05). The black circles indicate the cerebral atrophy in entorhinal and para-hippocampal regions and the red circles indicate the cerebral atrophy in precuneus region (FIG. 2A). The average cortical thickness in the medial temporal cortex (entorhinal and para-hippocampal regions) (FIG. 2B) and precuneus FIG. 2C) was compared between APOE genotypes (e4/4 and e3/3) in East Asian and Caucasian. Data were normalized to e3/3 and shown as percentage with error bars representing 95% confidence interval indicated above the bar plot. The bar graph shows the comparison of hippocampal volumes between APOE genotypes (e4/4 and e3/3) in East Asian and Caucasian (FIG. 2D).

FIG. 3 shows T-allele of rs405509 exhibiting the reduced expression of APOE protein. Human serums were subjected to Western blotting with anti-APOE and anti-TF (transferrin) to investigate rs405509 genotype-dependent serum expression levels of APOE protein (FIG. 3A). The serum samples were selected for the genotypes of rs405509 (G/G, G/T, and T/T) among APOE e3/3 homozygotes. The bar graph shows the relative intensity of APOE expression (FIG. 3B). The GG genotype was used as reference. Transferrin (TF) was used as a normalized control. Data represent means±SEM. (*p<0.05, ***p<0.001).

FIG. 4 shows graphs explaining Asian is more susceptible to APOE e4/4-mediated onset of Alzheimer's disease. The odds ratios of APOE e4/4 with reference to e3/3 for Alzheimer's disease were compared among different ethnic groups. The bar plots show the odds ratios of e4/4 for clinic diagnosis-based Alzheimer's disease (FIG. 4A) and neuropathological confirmed series (FIG. 4B). *Datasets were analyzed by logistic regression in this study. †The odds ratios were referred from the previous studies.

### BEST MODE

In the present invention, we, inventors investigated APOE e4-mediated AD susceptibility for different ethnic groups through large-scale study, showing that the e4 homozygotes from East Asian are more vulnerable to Alzheimer's disease rather than those from Caucasian and African ancestry. Further, we, inventors confirmed this ethnic difference by the association study with neuropathologically diagnosed Alzheimer's disease patients and controls, which is the first study up to date. A few studies reported the differential allele distribution of APOE e4 in diverse populations (Farrer et al., 1997; Singh et al., 2006). The geographic regional order of high frequency in e4 allele is the order of Africa, Europe, and Asian, which is exactly the reverse order of risk of e4 for Alzheimer's disease.

Lots of studies have reported that APOE e4 accelerates the cortical thinning in regions of the entorhinal cortex, parahippocampal cortex, and precuneous (Donix et al., 2010; Foley et al., 2016; Thompson et al., 2011). Another study on cognitively normal subjects, e4 carriers also showed decreased cortical thickness (Fouquet et al., 2014). In addition, the hippocampus, which plays a critical role in memory functions, has been reported to show the severe atrophy and memory dysfunction in individuals carrying e4 allele (Alexopoulos et al., 2011). In the present study, our analyses revealed that the APOE e4 homozygotes are associated with the severe cortical thinning in the areas of entorhinal, parahippocampal and precuneus regions and hippocampal atrophy in both East Asians and Caucasians relative to APOE e3 homozygotes, consistent with the previous studies (Donix et al., 2010; Foley et al., 2016; Thompson et al., 2011). Surprisingly, this cortical and hippocampal atrophies observed in e4/4 subjects were found to be more severe in East Asians rather than Caucasians, which is the unique aspects in this study. It is also matched with our association study. Therefore, the present invention suggests that the East Asians carrying APOE e4/4 are at increased risk towards not only Alzheimer's disease, but also cortical thinning and hippocampal atrophy relative to Caucasians.

We, inventors, hypothesized that the difference in e4/4-mediated AD risk between populations might be in part from the ethnic difference in genetic background, especially the polymorphisms in and around APOE gene. APOE e4 is not the only Alzheimer's disease-related APOE polymorphisms. It has been reported that the polymorphisms within APOE promoter and intron region modulate the effects of APOE e4 on the AD occurrence (Bertram et al., 2007; Lambert et al., 2002; Lambert et al., 1998b; Lescai et al., 2011). Two SNPs (rs449647 and rs405509) in the promoter and one SNP (rs440446) in intron 1 have been assessed for modulating the effect of APOE epsilon variants for Alzheimer's disease. The −491AA genotypes and the −219TT genotype have been reported to increase AD risk, independently of APOE epsilon genotypes (Lambert et al., 2002; Lambert et al., 1998a; Lambert et al., 1998b; Limon-Sztencel et al., 2016; Wang et al., 2000). In addition, rs405509 has been reported to synergizes with APOE e4 for the effects on cognitive ability (Ma et al., 2016). Here, we addressed that these SNPs showed the diverse allele frequencies between different ethnic populations. Among these SNPs, for rs405509, it has been confirmed that East Asians including Korean and Japanese have higher allele frequency of risk allele, rs405509-T, compared to Caucasian and African ancestry in both of whole population and e4 homozygote groups, possibly supporting that e4 homozygotes from East Asian have a high risk for Alzheimer's disease compared to other ethnic groups. In the same manner, it is confirmed that Caucasian has the higher allele frequency for rs405509-T rather than African ancestry along with that Caucasian has the higher odds ratio for e4/4 genotypes rather than African ancestry.

Even though several neuroimaging studies have reported the effects of APOE e4 homozygotes in brain atrophy, the effects of APOE promoter polymorphisms are not clearly studied until now. A previous study reported the effects of rs405509 polymorphism in APOE promoter region on the human brain during nondemented aging among Chinese population. It was found that rs405509-TT carriers with APOE e4 allele exhibited an accelerated age-dependent atrophy in cortical thickness relative to rs405509 G-allele carriers (Chen et al., 2015a). We, inventors, showed that the individuals with rs405509-TT genotype are highly associated with Alzheimer's disease and cause the stronger atrophy in cortical thickness and hippocampal volume compared to the carriers with G-allele among e4 homozygotes. Specifically, these cortical thinning patterns were observed in medial temporal cortex (entorhinal and parahippocampal regions) and precuneous, in which the atrophy in the parahippocampal region is in parallel with a previous study (Chen et al., 2015a). We, inventors, confirmed severe cortical thinning in the entorhinal, parahippocampal, and precuneus regions and the hippocampal atrophy among rs405509-TT carriers with APOE e4 homozygotes. Further, we confirmed that the ethnic difference in e414-mediated risk for Alzheimer's disease and brain atrophies results from the ethnic diversity in frequency of rs405509.

In the present invention, we, inventors demonstrated that rs405509-TT induced the reduced APOE expression in human serum and brain. Finally, in reporter gene assay with the allelic replacement in rs405509 T or G allele, we proved that APOE gene with rs405509-T allele in the promoter was less expressed, suggesting that the risk involving rs405509-T would be from the reduced APOE protein.

In this regard, it was observed that the APOE e4 homozygotes from East Asians were more vulnerable to the onset of Alzheimer's disease rather than those from Caucasian and African ancestry. The different allele frequencies in APOE promoter polymorphism among ethnic groups would make APOE e4 carriers differentially susceptible to onset of Alzheimer's disease. Given the involvement of rs405509 in regulation of the level of APOE protein, it is suggested that the reduced APOE level in e4 carriers with rs405509-TT causes the increased risk for Alzheimer's disease.

Hereinafter we explain the present invention by concrete Examples. These Examples are shown only for the purpose of explanation of the invention and the present invention is by no means limited by them.

### Mode for Invention

### Materials and Methods

### (1) Sample and Data

The information on full length genomes, MRI brain images, and clinical diagnosis was employed in the present invention. The full-length genome information was obtained from blood samples collected from 2,075 subjects for dementia study (Chosun University and its cooperative hospitals, normal subjects' average age: 73.45±5.30 (mean±SD), AD's disease patients' average age: 71.65±5.65 (mean±SD), females in normal subjects: 62.4%; females in AD's disease patients: 61.5%), and the information on brain images of each individual subject was obtained by MRI scanning. Neuropsychological Assessments (for example, K-MMSE and SNSB (Seoul Neuropsychological screening battery)) necessary for dementia diagnosis were carried out for each subject for study, and normal, MCI or dementia was diagnosed by a dementia specialist based on the results of the said assessments. According to the assessments results, 1,145 subjects were diagnosed as normal and 930 subjects were diagnosed as dementia patients. In addition, the data from the National Research center for Dementia (NRCD) in Gwang Ju, Korea were used for this study. All studies and research protocols were approved by the institutional review board of Chosun University Hospital, Korea. All volunteers and the relatives of patients gave written informed consent before participation. The subjects for East Asian consisted of 2,309 cognitively normal individuals and 1,886 AD patients. The Caucasian subjects' genomes and brain images were obtained from ADNI (Alzheimers Disease Neuroimaging Initiative; http://adni.loni.usc.edu/) database. The subjects for Caucasian consisted of 515 cognitively normal and 320 with AD.

The clinical diagnosis of AD was performed in accordance with the AD criteria of the National Institute of Neurological and Communication Disorders and Stroke/Alzheimer's Disease and Related Disorders Association (NINCDS-ADRDA) (McKhann et al., 1984b). MCI was diagnosed according to the current consensus criteria (Winblad et al., 2004). The cognitively normal group had no neurological disease and no impairment in cognitive functions or activities of daily living. The exclusion criteria for all subjects were presence of a focal lesion on brain MRI, history of head trauma, or psychiatric disorders that could affect their mental function. Individuals with minor medical abnormalities (e.g., essential hypertension, diabetes without serious complications, or mild hearing impairment) were included.

The Caucasian genomes from data sets of NIA-LOAD (National Institute on Aging-late onset Alzheimer's disease), NCRAD (National Cell Repository for Alzheimer's Disease), or ADNI (Alzheimers Disease Neuroimaging Initiative) were used to study odds ratio of rs405509. APOE promotor single nucleotide of the subjects having APOE E4/E4 genotype was used for the study. A total of 395 subjects from data sets of NIA-LOAD, NCRAD and ADNI had E4/E4 genotype from the data sets of NIA-LOAD, NCRAD and ADNI, and among them, 300 subjects were AD patients and 65 subjects were cognitively normal.

### (2) Brain Amyloid PET Imaging

¹⁸F-Florbetaben PET imaging was performed as described previously (Osama Sabri, 2015, ClinTransl Imaging, Barthel, 2011, Lancet neurology). Amyloid pathology was determined by the brain amyloid-β plaque load (BAPL) value. The experts assessed the florbetaben (18F) PET images according to a predefined regional cortical tracer uptake (RCTU) scoring system (1=no uptake, 2=minor uptake, 3=pronounced uptake) for 4 brain regions (frontal cortex, posterior cingulate, lateral temporal cortex, parietal cortex). The RCTU scores for the frontal cortex, posterior cingulate, lateral temporal cortex, and parietal cortex were then summarized into a single predefined three-grade scoring system for each PET scan, the brain amyloid-6 plaque load (BAPL) score (1=no amyloid load, 2=minor amyloid load, 3=significant amyloid load). In vivo amyloid imaging via positron emission tomography (PET) with Pittsburgh compound B (PiB) was conducted. Deposition of PiB in brain regions of interest was determined using FreeSurfer version 5.1 software (Martinos Center for Biomedical Imaging), and a standardized uptake value ratio (SUVR) corrected for partial volume effects was calculated for each region of interest. The mean cortical SUVR was calculated from FreeSurfer regions within the prefrontal cortex, precuneus, and temporal cortex. Cerebellar cortex served as the reference region. PiB positivity was defined as an SUVR of 1.42, which commensurates with a mean cortical binding potential of 0.18 defined previously for PiB positivity. The data of amyloid-PET for Caucasian were obtained from the ADNI database (http://adni.loni.usc.edu) for both 18F-florbetapir and 11C-PiB.

### (3) Data Generation and Analysis

### The SNP Genotyping

Genomic DNAs were extracted peripheral blood leukocytes isolated from whole blood cells collected in EDTA tubes. After blood samples were centrifuged at 1500×g for 10 minutes, the plasma was removed, and the blood leukocytes were used for DNA extraction. The samples were genotyped using Affimetrix Genome-wide genotyping arrays (Affymetrix® Axiom KORV1.0), which was optimized for Korean. The KORV1.0 chip was available through the Korean chip consortium, and designed by Center for Genome Science, Korea National Institute of Health, Republic of Korea (4845-301, 3000-3031). In addition, the genotyping assays through the Taqman or SNP type (Fluidigm) methods were conducted for the evaluation of accuracy in chip-based SNP genotyping. Genotype data for ADNI were obtained from the ADNI database (http://adni.loni.usc.edu).

### Imputation of Genome Wide Data

The study data set from NRCD consisted of 6,413 individuals. The data set from ADNI consisted of 818 individuals from ADNI-1, 432 from ADNI-GO/2, and 818 from ADNI-WGS data sets. Quality control for samples included individual call-rate <95%, gender inconsistencies, heterozygosity (±3SD from mean), duplicates or twins, SNPs with call-rate <95%, SNPs with HWE P-value <10⁻⁶ and MAF <1%. Each data sets were imputed separately using HRC reference panel version 1.1. The HRC imputation from NRCD was run by selecting East Asians as reference population with pre-phased study haplotypes. ADNI datasets were run separately by selecting Europeans as reference population in HRC imputation server with pre-phased study haplotypes. After imputation, the low-quality imputed SNPs (info<0.5) were removed for further analysis (McCarthy et al., 2016; Nagy et al., 2017; Surakka et al., 2016).

### (4) Imaging Protocol

MR Images from Korean Population

For the 2443 Korean subjects, contiguous 0.9 mm axial MPRAGE images of the whole brain were acquired using the 1.5 T MR scanner (Magnetom Avanto, Siemens) with TR=1800 ms; TE=3.43 ms; TI=1100 ms; 15 flip angle; FoV=224×224; matrix=256×256; number of slices=176. The contiguous 0.8 mm sagittal MPRAGE volumes were acquired using the 3 T MR scanner (Skyra, Siemens), with TR=2300 ms; TE=2.143 ms; TI=900 ms; 9 flip angle; FoV=256×256; matrix=320×320; number of slices=178. All the images acquired from the MR scanners located at Chosun University Hospital, Gwangju, South Korea.

MR Images from ADNI

The Alzheimer's disease Neuroimaging initiative (ADNI) is a large longitudinal multi-centre based observational study consisting of cognitively normal, individuals with MCI and AD (Jack et al., 2008). In this study, the baseline 1.5T and 3T MR images from 463 cognitively normal subjects, 796 subjects with mild cognitive impairments, and 310 subjects with AD were downloaded from the ADNI database (ADNI, http://adni.loni.usc.edu/). All MR images for the subjects were collected on 1.5T or 3T GE, Philips and Siemens scanners using standard ADNI MPRAGE protocol. In 1.5T scanners, the nominal parameters for MPRAGE protocol were sagittal plane, TR=2300-2400 ms for multi-coil phased-array head coil (TR=3000 ms for bird cage or volume head coil), minimum full TE, TI=1000 ms, flip angle 8°, FOV=240×240 mm, 192×192 in-plane matrix and slice thickness 1.2 mm. In 3T scanners, the MPRAGE protocol were sagittal plane, TR=2300 or 3000 ms, minimum full TE, T1=853-900 ms, flip angle 8-9°, FOV=256-260×240 mm, 256×256 in-plane matrix and slice thickness 1.2 mm (Jack et al., 2008). The AD subjects were included based on 20-26 scores on the MMSE (Burns et al., 1998) and CDR=0.5 or 1 (Morris, 1993). In addition, the selected AD groups also met the NINCDS/AARDA criteria for probable AD (McKhann et al., 1984a). For the healthy controls, CDR of 0 and the non-demented individuals were included (Wolz et al., 2011).

### (5) Image Data Processing and Analysis

### MR Image Data Processing

Image processing was performed using FreeSurfer software (FreeSurfer 5.3.0), a widely used and publicly available software automated for the brain structure analysis (FreeSurfer, http://surfer.nmr.mgh.harvard.edu/) (Dale et al., 1999; Fischl and Dale, 2000; Fischl et al., 2004). The MR images in DICOM format were used in the automated pipeline for the FreeSurfer processing to construct a 3-dimensional model of cortical surface (Dale et al., 1999; Dale and Sereno, 1993; Fischl et al., 1999a). Briefly, in this process, the intensity of the image was normalized, and the skull was stripped from the normalized image. Then the connected components algorithm initiated the subcortical segmentation process, and any holes found were filled to represent a single-filled volume of white matter for both the cortical hemispheres. Furthermore, tessellation and surface smoothening were employed to decrease any metric distortions. Then, refinement process was performed to yield a gray/white matter boundary, followed by initial surface model construction. Subsequently, the surface was deformed outwards to construct the pial surface. Cortical thickness measurements were obtained by first calculating the shortest distance to the pial surface from each point on the white/gray matter surface and from the point of pial surface, the shortest distance to gray/white matter was calculated, and then the final cortical thickness was obtained from the average of these two values at that specific point. Cortical thicknesses for 163,842 vertices in each hemisphere were obtained by using triangular grid spaced approximately 1 mm apart from any point or other sites representing vertex in the cortical mantle. The Cortical folding patterns were aligned by smoothening the maps with a full-width-half-maximum Gaussian kernel of 0, 5, 10, 15, 20 and 25 mm and averaging across the subjects using a non-rigid high-dimensional spherical averaging method. The aligned maps were available for detecting submillimeter differences among the subjects and not limited to the voxel resolution. Subcortical volumes were also obtained through automated method for volumetric measures using FreeSurfer (Dale et al., 1999; Fischl and Dale, 2000; Fischl et al., 1999b).

### (6) Statistical Analysis for Imaging

Cortical thickness differences between different APOE allele carrier groups were statistically analyzed using Surfstat (http://www.math.mcgill.ca/keith/surfstat/) toolbox for MATLAB (R2012a, The Mathworks, Natick, Mass., USA). We tested each unmasked points on the pial surface statistically. General linear model (GLM) was applied to check the point-wise cortical thickness differences using APOE alleles (e4/4 vs e3/3) as fixed factor, and sex, age, education and field strength as covariates (Fan et al., 2010). For the evaluation of rs405509 allele differences, T/T-E4/4 and G/T-e4/4+G/G-e4/4 haplotype carriers were compared with e3/3 carriers separately. In the GLM of rs405509, we checked the point-wise cortical thickness differences using APOE alleles (rs405509-e4/4 haplotype vs e3/3) as fixed factor, and sex, age, education and fieldstrength as covariates (Chen et al., 2015b). In all cortical thickness analyses, a random field theory (RFT) based correction for multiple point-wise cortical thickness comparisons were applied at the cluster level, and the clusters passing false discovery rate (FDR) correction at p<0.05 were considered as significant (Taylor and Adler, 2003) (http://www.math.mcgill.ca/keith/surfstat/). In addition, hippocampal volumes and the anatomical region of interest (ROls) were statistically compared between APOE allele carrier groups in the R program (R version 3.3.1). Hippocampal volume differences were assessed by the analysis of covariance (ANCOVA) with APOE alleles (e4/4 vs e3/3, rs405509-e4/4 haplotype vs e3/3) as fixed factor, and sex, age, education, diagnosis and intra cranial volume (ICV) as covariates (Hickie et al., 2005; Zierhut et al., 2013). ROI differences were then compared between different groups (e4/4 vs e3/3, rs405509-e4/4 haplotype vs e3/3) with age, sex, education and diagnosis as covariates. In these analyses, the significance level was considered at p<0.05 and the findings were considered significant if surviving FDR correction at the p<0.05 level (Stricker et al., 2013; Ye et al., 2016).

### (7) Statistical Analysis

The proportion of subjects in control group and AD group were compared with chi-square tests. The odds ratios for events are calculated using logistic regression with 95% confidence interval for both adjusted (age and sex) and unadjusted analysis (Basaria et al., 2010). All the statistical tests were performed using both SPSS (version 23.0) and R program (version 3.3).

(8) APOE Gene Expression from Human Blood Samples

### Western Blotting

First, serums were collected from the whole blood samples (obtained from NRCD). SDS-PAGE and Western blotting were performed as described by the manufacturer (Amersham Biosciences, Piscataway, N.J.). Briefly, membranes were blocked with 5% nonfat dry milk in PBS with 0.1% Tween 20 for 1 hour and subsequently incubated with primary antibodies for 1 hour at room temperature. Primary antibodies were used at the following dilutions: 1:1,000 rabbit anti-APOE (D719N; Cell Signaling); 1:1,000 rabbit anti-transferrin (ab109503; Abcam). Horseradish peroxidase-conjugated secondary antibodies were used at 1:5,000. Immuno-reactivity was detected with an EZ-Western Lumi Plus (DoGen).

### (9) Reporter Gene Assays

APOE promoter construct—Promoter region of APOE gene (positions −1,983 to +935) was amplified using genomic DNA obtained from human normal or AD patient blood cell using the following primers: forward, 5′-GGGGTACCGAAAGCAGCGGATCCTTGAT-3′ (SEQ ID NO.1); reverse, 5′-CCCCTCGAGCTTCCTGCCTGTGATTGGC-3′ (SEQ ID NO.2). The amplified DNA was digested with Kpnl and Xhol and ligated into the pGL3.basic vector (Promega). PCR based Site-directed mutagenesis of rs405509 (−219G/T) was carried out to replace G→T and T→G allele using the following primers: T→G forward, 5′-GAGGAGGGTGTCTGGATTACTGGGCGAG-3′ (SEQ ID NO.3); reverse, 5′-CTCGCCCAGTAATCCAGACACCCTCCTC-3′ (SEQ ID NO.4) G→T; forward, 5′-GAGGAGGGTGTCTGTATTACTGGGCGAGG-3′ (SEQ ID NO.5); 5′-CCTCGCCCAGTAATACAGACACCCTCCTC-3′ (SEQ ID NO.6). The above analyses were performed using PfuUltra High-Fidelity DNA Polymerase (Agilent).

### (10) Luciferase Assay

HEK 293T cells were cultured in 12-well plates. After 24 h, the cells were co-transfected with 0.25 μg of pGL3 carrying firefly luciferase reporter gene (Promega) and 0.25 μg of pCMV-β-galactosidase (Clontech) using TransFectin™ Lipid Reagent for 24 hours. Transfected cells were lysed with reporter lysis buffer (Promega). Luciferase and β-galactosidase activities were quantitated by using a GloMax® Luminometer (Promega) and Epoch microplate spectrophotometer (BioTek), respectively. Luciferase activity was normalized by β-galactosidase activity. Results were obtained from three independent experiments.

### (11) Information on Full-Length Genome

To obtain information on full-length genome, buffy coats isolated from blood samples independently collected from cognitively normal subjects, MCI patients and AD's dementia patients were used. The gDNAs were extracted using QIAGEN DNA mini kit and then purified. Then, quantification was conducted with ND-1000 spectrophotometer and Quant-iT PicoGreen dsDNA Reagent and Kits. Subsequently, DNA quality was confirmed by electrophoresis on agarose 1.0% TBE (Tris-borate-EDTA, Invitrogen) gel, and then genotype analysis based on microarray was carried out for the samples satisfying above DNA quality controls. The information on each genotype was obtained using Axiom Korean chip (Affymetrix). Imputation was conducted for SNPs, which were not microarrayed, to determine genotype thereof. Programs, such as, plink, EIGENSTRAT, Shapeit, impute2, GTOOL, EAGLETOOL, and minimac3 were used for full-length genome imputation, and 1000 Genomes (https://www.ncbi.nlm.nih.gov/variation/tools/1000genomes/) and Michigan imputation server (https://imputationserver.sph.umich.edu/index.html) were used as reference panels.

### Results

Association of APOE e4/4 with Alzheimer's Disease

In an association study for Alzheimer's disease, the risk of APOE variants among different ethnic groups of East Asian, Caucasian, and African ancestry was determined. In all ethnic groups with control groups and clinically diagnosed Alzheimer's disease patients, the distribution of APOE genotypes and allele frequencies showed significant difference between control groups and Alzheimer's disease patients. There were more e4-carriers in Alzheimer's disease group rather than control groups (data not shown). Even though APOE e4 is a critical risk factor for Alzheimer's disease, the ethnic difference in e4-mediated risk has not been well addressed. In an analysis using the patients clinically diagnosed with probable Alzheimer's disease and normal control groups, the e4/4 subjects from East Asian showed the significantly higher odds ratio rather than those of Caucasian (Odds ratios for+e4/4=33.40 and 15.86 for East Asian and Caucasian, respectively) (Table 1). In Table 1, the Odds ratios were calculated with reference to e3/e3 by Logistic Regression.

TABLE 1

| | | | OR (95% C.I.) | | |
|---|---|---|---|---|---|
| Diagnosis | Population | N | e3/4 | e4/4 | Ref. |
| Clinically diagnosed | | | | | |
| | East Asian | | | | |
| | East Asian* | 4195 | 3.94 | 33.40 | |
| | (NRCD) | | (3.4-4.6) | (17.0-65.8) | |
| | Korean† | 336 | 2.9 | 24.7 | Kim KW, 1999 |
| | | | (1.7-5.2) | (2.8-214.5) | |
| | Japanese† | 2313 | 5.6 | 33.1 | Farrer LA, |
| | | | (3.9-8.0) | (13.6-80.5) | 1997 |
| | European ancestry | | | | |
| | Caucasian*,‡ | 835 | 3.81 | 15.86 | |
| | (ADNI) | | (2.73-5.3) | (8.17-30.77) | |
| | Caucasian† | 6305 | 3.2 | 14.9 | Farrer LA, |
| | (Clinic/Autopsy) | | (2.8-3.8) | (10.8-20.6) | 1997 |
| | Caucasian† | 4858 | 2.7 | 12.5 | Farrer LA, |
| | (population-based) | | (2.2-3.2) | (8.8-17.7) | 1997 |
| | France† | 1447 | 2.2 | 11.2 | Bickeboller H, |
| | | | (1.5-3.5) | (4.0-31.6) | 1997 |
| | Norway† | 937 | 4.2 | 12.9 | Sando SB, |
| | | | (3.0-5.9) | (6.6-26.7) | 2008 |
| | African ancestry | | | | |
| | African-Americans† | 474 | 1.1 | 5.7 | Farrer LA, |
| | | | (0.7-1.8) | (2.3-14.1) | 1997 |
| | Nigeria† | 582 | 1.33 | 1.68 | Gureje O, |
| | | | (0.8-2.1) | (0.7-3.7) | 2006 |
| | Tunisia† | 129 | 2.9 | 5.4 | Rassas AA, |
| | | | (1.3-6.6) | (1.4-21.5) | 2012 |
| Neuropathologically confirmed | | | | | |
| | East Asian*,§ | 883 | 5.26 | 125.10 | |
| | (NRCD) | | (3.7-7.4) | (16.7-935.2) | |
| | Caucasian*,¶ | 1067 | 6.48 | 20.92 | |
| | (ADNI) | | (4.7-8.9) | (10.2-48.9) | |

*The odds ratios were analyzed with reference to e3/3 by logistic regression in the present study;
†The odds ratios were referred from the previous studies stated in rightmost column;
‡The datasets from Alzheimer's Disease Neuroimaging Initiative (ADNI) were analyzed for the risk for Alzheimer's disease in Caucasian;
§Amyloid burden was assessed for subjects of NRCD for neuropathological diagnosis;
¶The data of Caucasian for amyloid accumulation were from the Alzheimer's Disease Neuroimaging Initiative (ADNI).

It is surprising that e4/4-mediated risk is ethnic-group dependent. The East Asian is more vulnerable than the Caucasian. The results are consistent with the previous studies analyzing the association of APOE genotypes for Alzheimer's disease (Table 1). Table 2 hereunder represents demographic characteristics of subjects in Alzheimer's disease and control groups based on clinical diagnosis. *

TABLE 2

| | | Cognitively normal controls | | | Alzheimer's disease cases | | |
|---|---|---|---|---|---|---|---|
| Population | Total | n | Female (%) | Age† (mean ± SD) | n | Female (%) | Age‡ (mean ± SD) |
| East Asian§ | 4195 | 2309 | 1423 (61.6%) | 74.6 ± 5.7 | 1886 | 1288 (68.3%) | 73.6 ± 5.6 |
| (NRCD) | | | | | | | |
| Caucasian | 835 | 515 | 265 (51.4%) | 74.38 ± 5.58 | 320 | 139 (43.4%) | 75.65 ± 6.84 |
| (ADNI) | | | | | | | |

* The age of elderly persons were 60 years or more and less than 90;
†The age for controls means age at examination;
‡The age for Alzheimer′s disease means age at onset;
§The dataset of East Asian was from National Research Center for Dementia (NRCD);
¶ The dataset of Caucasian was from the Alzheimer′s Disease Neuroimaging Initiative (ADNI).

To confirm the ethnic difference for e4/4-mediated onset, the association was analyzed using datasets of neuropathological diagnosis from Korean and Caucasian (Table 3).

TABLE 3

| | | Ab (−) † | | | Ab (+) † | | |
|---|---|---|---|---|---|---|---|
| | | | | Age‡ | | | Age§ |
| Population | Total | n | Female (%) | (mean ± SD) | n | Female (%) | (mean ± SD) |
| East Asian¶ | 883 | 557 | 321 (55.6) | 72.3 ± 6.3 | 306 | 162 (52.9) | 73.4 ± 6.0 |
| (NRCD) | | | | | | | |
| Caucasian∥ | 1067 | 475 | 203 (42.7) | 72.8 ± 6.5 | 592 | 266 (44.9) | 74.3 ± 6.3 |
| (ADNI) | | | | | | | |

* The age of elderly persons were 60 years or more and less than 90;
† Ab (−) indicate amyloid-negative and Ab (+) indicate amyloid-positive;
‡The age for controls means age at examination;
§The age for Alzheimer′s disease means age at onset;
¶East Asian were Korean participated in amyloid-PET scan;
∥The dataset of Caucasian for amyloid pathology were from Alzheimer′s Disease Neuroimaging Initiative (ADNI).

In both ethnic groups, there were more e4 carriers in amyloid-positive group rather than negative group (Table 4).

TABLE 4

| | APOE | | Genotypes frequenies (%) | | | | | | Allele frequencies (%) | | |
|---|---|---|---|---|---|---|---|---|---|---|---|
| | Genotype | n | e2/2 | e2/3 | e2/4 | e3/3 | e3/4 | e4/4 | e2 | e3 | e4 |
| East Asian | Aβ (−)* (%) | 577 | 0.3 | 10.4 | 0.5 | 74.3 | 14.2 | 0.2 | 5.8 | 86.6 | 7.6 |
| (NRCD) | Aβ (+)* (%) | 306 | 0.6 | 2.6 | 1.6 | 42.8 | 42.5 | 9.8 | 2.8 | 65.4 | 31.9 |
| | γ2 (P) | | 180.60 (4.010⁻³⁷) | | | | | | 178.33 (1.810⁻³⁹) | | |
| Caucasian | Aβ (−) (%) | 475 | 0.4 | 13.7 | 1.1 | 66.1 | 17.1 | 1.7 | 11.5 | 73.5 | 15.0 |
| (ADNI) | Aβ (+) (%) | 592 | 0 | 2.5 | 2.9 | 33.4 | 47.4 | 13.2 | 3.5 | 54.8 | 41.7 |

| | | |
|---|---|---|
| γ2 (P) | 203.05 (4.51 × 10⁻⁴⁶) | 122.16 (2.1 × 10⁻²⁸) |

*Ab (−) indicate amyloid-negative subjects and Ab (+) indicate amyloid-positive subjects.

In the association study between APOE genotypes and amyloid accumulation, the e4/4 subjects in Korean showed dramatically higher odds ratio compared to those in Caucasian (Odds ratios for e4/4=125.1 and 20.92 for Korean and Caucasian), consistent with the result from clinically diagnosed subjects (Table 1 and Table 5). These results suggest that e4 homozygotes from the East Asian are more vulnerable to Alzheimer's disease rather than those from other ethnic groups.

TABLE 5

| | | | e2/2 + e2/3 | | e2/4 | | e3/4 | | e4/4 | |
|---|---|---|---|---|---|---|---|---|---|---|
| Population | n | e3/3† | OR‡ (95% C.I.) | P | OR (95% C.I.) | P | OR (95% C.I.) | P | OR (95% C.I.) | P |
| East Asian | 883 | Ref | 0.49 | 0.049 | 5.33 | 0.024 | 5.26 | 1.910⁻²¹ | 125.10 | 3.010⁻⁶ |
| (NRCD) | | | (0.2-0.9) | | (1.2-22.8) | | (3.7-7.4) | | (16.7-935.2) | |
| Caucasian | 1067 | Ref | 0.38 | 0.0015 | 6.27 | 0.0005 | 6.46 | 2.810⁻³⁰ | 20.92 | 1.310⁻¹⁴ |
| (ADNI) | | | (0.2-0.7) | | (2.4-19.7) | | (4.7-8.9) | | (10.2-48.9) | |

* The association between APOE genotypes and amyloid-based neuropathological diagnosis was tested in each ethnic group by logistic regression;
†e3/3 was used for reference genotype;
‡The odds ratios were adjusted with gender and age.

### Screening the Polymorphisms in and Around APOE Gene

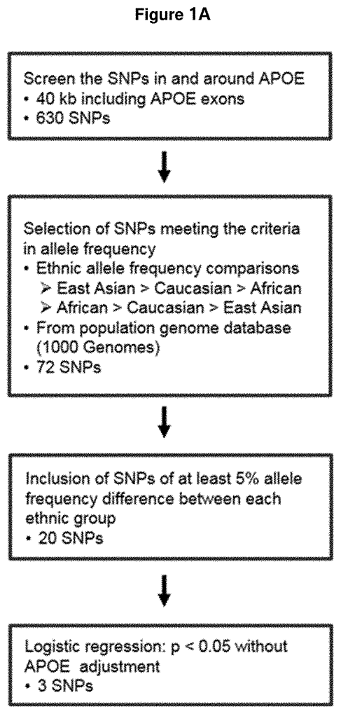

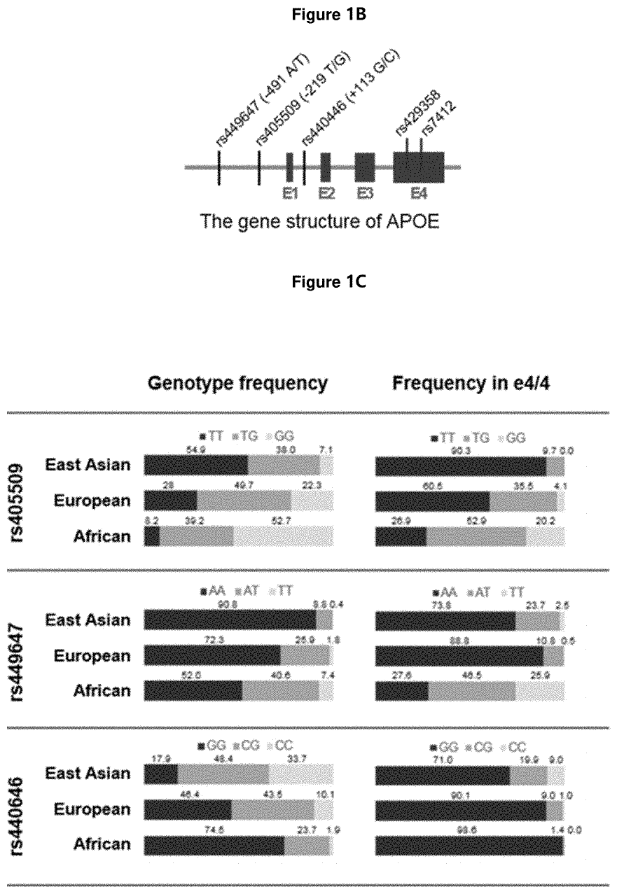

One speculation is that the differential risk of e4/4 among ethnic groups with the risk order of East Asian, Caucasian, and African ancestry for Alzheimer's disease would be from the ethnic difference in aspects of the polymorphisms in and around APOE gene. To investigate the causal mechanism, we screened the SNPs in a 40 kb region surrounding APOE gene for the allele frequencies among different ethnic groups (FIG. 1A). Among 630 imputed SNPs across 40 kb, 72 SNPs were elected in accordance with the criteria that the allele frequency is the order of East Asian, Caucasian, and African ancestry, or vice versa. Subsequently, we cut off the SNPs by minor allele frequency and the significance of association with Alzheimer's disease without APOE adjustment. Finally, there remained three SNPs, rs449647, rs405509, and rs440446. These polymorphisms are located at APOE promoter and intron region (FIG. 1B). They show the big difference in risk allele frequency (rs449647-A, rs405509-T, and rs440446-G) between East Asian, Caucasian, and African ancestry (FIG. 1C). Among them, rs405509 shows the dramatic difference in allele frequencies between ethnic groups (Based on 1000 genome database, T-allele frequency=0.67, 0.48, and 0.24 for East Asian, Caucasian, and African ancestry, respectively). In particular, e4 homozygotes from East Asians have over 90.4% TT-genotypes, while Caucasian and African ancestry have 64.2% and 21.3% of TT-genotypes, respectively, suggesting that the differences in allele frequencies of rs405509 may confer differential susceptibilities to AD onset for e4/4 of different ethnic groups. Even though rs449647 and rs440446 showed the differential allele frequency among different ethnic groups, their frequencies in e4/4 are little different among ethnic groups, taken out of our consideration.

Association of Rs405509 with Alzheimer's Disease

To investigate if TT genotype of rs405509 confers higher risk of Alzheimer's disease in e3/4 and e4/4, association of rs405509 among e3/4 and e4/4 subjects was tested. As expected, rs405509-TT showed significantly high odds ratio in both groups of e3/4 and e4/4 subjects from NRCD dataset (East Asian) (Table 6), providing a clue about the ethnic difference in risk of e4/4, in which e4 homozygotes from East Asian with high frequency of rs405509-TT are more susceptible to Alzheimer's disease rather than those from Caucasian and African ancestry.

TABLE 6

| | | | APOE | rs405509 in e3/4 (TT vs GT + GG) | | APOE e4/4 | rs405509 in e4/4 (TT vs GT + GG) | |
|---|---|---|---|---|---|---|---|---|
| Group | | Total(n) | e3/4(n) | OR† (95% C.I.) | P | (n) | OR† (95% C.I.) | P |
| NRCD | NP | 14322 | 2272 | 1.40 | 6.59 × 10⁻⁰⁵ | 140 | 3.59 | 4.48 × 10⁻⁰⁵ |
| | AD | 2079 | 855 | (1.19-1.65) | | 197 | (1.94-6.62) | |

†The odds ratios of rs405509-TT genotype were analyzed with reference to rs405509-GT + GG genotypes by logistic regression among e3/4 and e4/4 subjects;
‡NP denotes normal population controls;
§ AD denotes Alzheimer′s disease patients.

In addition, to confirm the effect of rs405509 on e3/4 and e4/4-mediated risk of Alzheimer's disease, we analyzed the association of e3/4 and e4/4 in different rs405509 genotype background, rs405509-TT, GT, and GG (Table 7). As results, e3/4 and e4/4 subjects showed higher odds ratio on the rs405509-TT genotypes rather than rs405509-G allele background in both NRCD and ADNI dataset.

TABLE 7

| | | | | e3/4 | | e4/4 | |
|---|---|---|---|---|---|---|---|
| Dataset | rs405509 | n | e3/3 | Odds ratio (95% C.I.) | P | Odds ratio (95% C.I.) | P |
| NRCD | TT | 8773 | ref | 4.23 (3.71-4.81) | 2.55E−105 | 15.53 (12.14-19.87) | 1.39E−105 |
| | GT | 7212 | ref | 3.69 (3.08-4.41) | 3.90E−46 | 7.50 (3.62-15.57) | 6.24E−8 |
| | GG | 7183 | ref | 2.09 (1.03-4.23) | 0.041 | 4.36 (0.18-107.99) | N/A |
| ADNI | TT | 265 | ref | 6.79 (3.67-13.03) | 2.80E−9 | 22.83 (7.89-85.42) | 1.54E−7 |
| | GT | 482 | ref | 5.03 (3.27-7.85) | 3.75E−13 | 11.85 (4.22-42.72) | 1.83E−5 |
| | GG | 197 | ref | 5.09 (2.09-13.63) | 5.73E−4 | N/A | N/A |

* The odds ratios of APOE genotypes e3/4 and e4/4 were analyzed with reference to e3/3 for Alzheimer′s disease by logistic regression among each rs405509 genotype dataset (each group of rs405509-TT, rs405509-GT, and rs405509-GG genotypes) for independent datasets.

Association of APOE e4/4 with Brain Atrophy

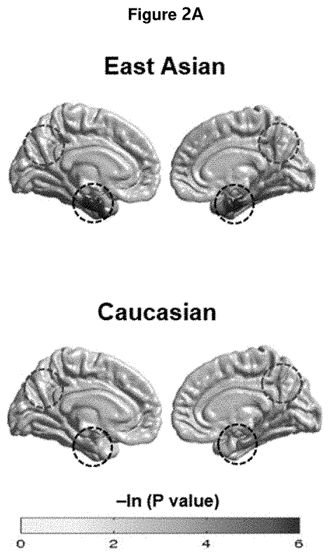

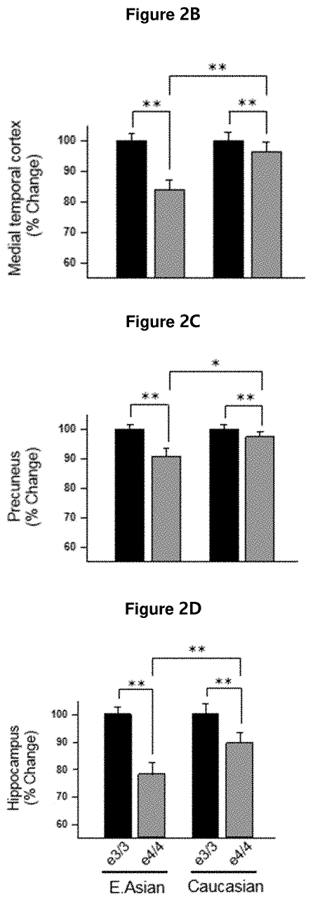

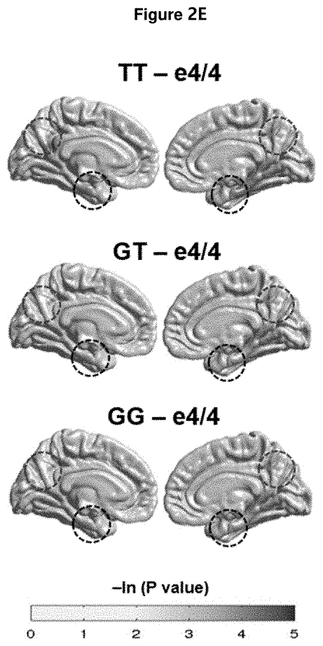

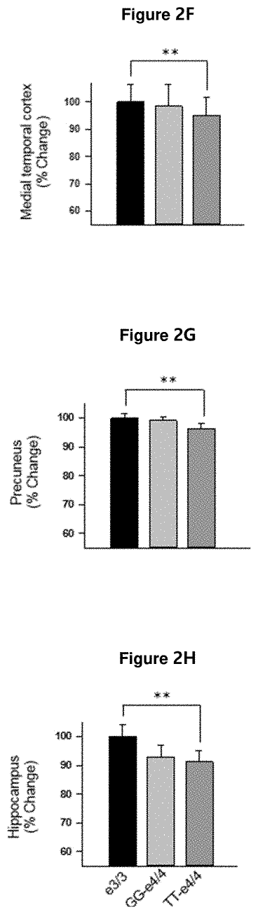

To check if the brain atrophy mediated by APOE e4/4 exhibits the ethnic difference, we analyzed the point-wise cortical thickness between e4/4 and e3/3 subjects for East Asian and Caucasian through general linear model. Both East Asian and Caucasian showed that e4/4 subjects exhibited the thinner cortical areas relative to e3/3 (FIGS. 2A, 2B, and 2C). The e4/4-dependent shrinkage regions were entorhinal-parahippocampal-fusiform, precuneus, and inferior parietal lobule (P<0.05). Surprisingly, the cortical thinning in East Asian was stronger than that in Caucasian in these areas. Analysis of covariance (ANCOVA) was conducted to measure the ethnic difference in e4/4-dirven cortical atrophy. The relative average cortical thickness in the cortical atrophy areas were calculated for e4/4-subjects with reference to e3/3. Both East Asian and Caucasian showed the significant difference between e4/4 and e3/3 in average cortical thickness in medial temporal cortex, including entorhinal cortex and parahippocampal cortex, and precuneus (*P<0.05, **P<0.01). In addition, East Asian displayed the bigger difference between e4/4 and e3/3 rather than Caucasian (FIGS. 2B and 2C). The direct comparison of relative cortical shrinkage of e4/4 (normalized to e3/3) between East Asian and Caucasian also shows the significance (*P<0.05, **P<0.01). In addition, we analyzed the hippocampal volume between e4/4 and e3/3 subjects in East Asian and Caucasian. Even though the hippocampal atrophy in e4/4 compared to e3/3 was significant in both East Asian and Caucasian (**P<0.01), the e4/4-driven hippocampal atrophy rate compared to e3/3 was bigger in East Asian rather than Caucasian (**P<0.01) (FIG. 2D). Taken together, our observations suggest that East Asian is more vulnerable to APOE e4/4-mediated brain atrophy including cortical thinning and hippocampal shrinkage. To examine the effect of rs405509 on brain atrophy among e4 homozygotes, we analyzed the cortical thinning for the combination of e4/4 and each genotype of rs405509 (e4/4-TT, e4/4-GT, and e4/4-GG) with reference to e3/3 in the Caucasian subjects (FIGS. 2E, 2F, and 2G). The e4/4 subjects with rs405509-TT showed the significant clusters in the medial temporal cortex (entorhinal and parahippocampal cortex) and precuneus, (P<0.05). Whereas, e4/4 subjects with rs405509-G allele showed no significant clusters. The quantitative comparison by ACNOVA analysis showed that e4/4 with r405509-TT genotype exhibited the significant atrophy in the medial temporal cortex (F=8.38, P<0.01) and precuneous (F=10.707, P<0.01) compared to e3/3, whereas e4/4 with rs405509-GG showed no significance in any of cortical regions (FIGS. 2F and 2G). We also tested the hippocampal atrophy of e4/4 subjects with rs405509-TT and rs405509-GG in Caucasian. The hippocampal shrinkage of e4/4 with rs405509-TT genotype was significantly stronger than that of e3/3, whereas the shrinkage of e4/4 with rs405509-GG was not significant (FIG. 2H). These neuroimaging data indicate that TT genotype in rs405509 has an effect on not only risk for Alzheimer's disease but also brain shrinkage in e4/4 subjects. Table 8 presents demographic characteristics of participants for neuro imaging.

TABLE 8

| | East Asian* | Caucasian† |
|---|---|---|
| | (NRCD) | (ADNI) |
| n | 1684 | 831 |
| Age§ (y, mean ± SD) | 72.77 ± 5.12 | 74.70 ± 6.59 |
| Female (n, %) | 1028 (61.0%) | 358 (43.0%) |
| Years of education | 9.01 ± 5.10 | 16.05 ± 2.78 |
| (y, mean ± SD) | | |
| MMSE (mean ± SD) | 26.01 ± 3.84 | 27.43 ± 2.54 |

*East Asian are the participants in National Research Center for Dementia (NRCD), Chosun University, Gwangju, South Korea;
†Caucasian are the participants in ADNI cohort;
§The age of elderly persons were 60 years or more and less than 90.

### The Expression of APOE

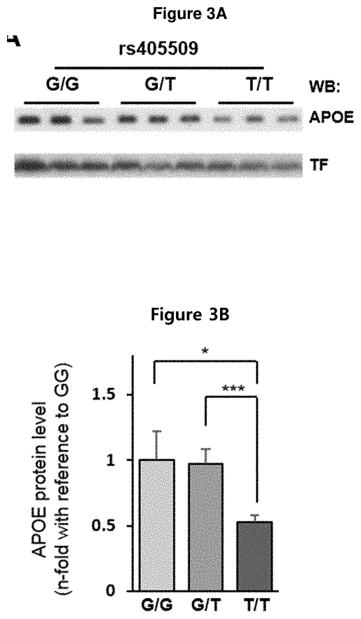

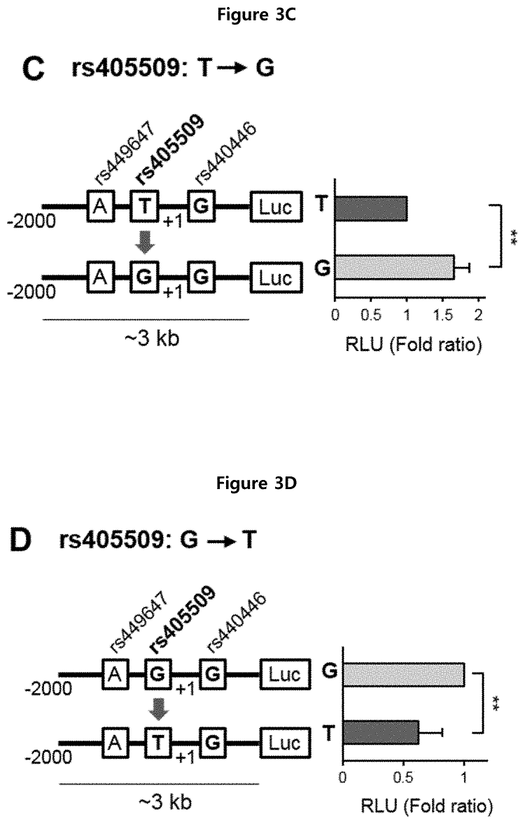

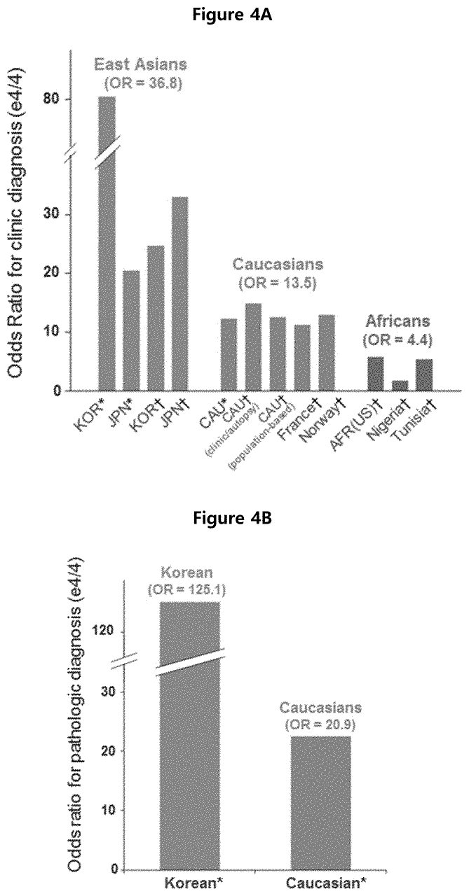

According to the present invention, rs405509 is associated with onset of Alzheimer's disease and brain atrophy among e4 homozygotes. Hence, we tried to evaluate the biological relevance of rs405509 for regulation of APOE. To examine if rs405509 genotypes regulate the expression of APOE protein, we conducted Western blotting for human sera of the genotypes of rs405509-TT, GT, and GG among e3/3 subjects (FIG. 3A). The level of APOE protein of subjects with rs405509-TT was dramatically reduced compared to those with rs405509-GG and GT (FIG. 3A). The result is matched with the previous study, suggesting that reduced levels of APOE is involved in the increased risk for Alzheimer's disease. In addition, the effect of rs405509 polymorphism in the transcriptional activity of APOE was analyzed by reporter gene assay. The assays were conducted with APOE promoter fragments from AD patient with rs405509-T allele and cognitively normal person with rs405509-G allele. The allele of rs405509 in each promoter region was changed to alternative-allele, and then subject to luciferase-based reporter gene assay (FIGS. 3C and 3D). A single T to G base substitution at rs405509 position caused a dramatic increase in APOE promoter activity (160% of control), whereas G to T substitution showed a dramatic decrease on promoter activity (66% of control), indicating that rs405509-T allele reduces APOE transcription compared to G allele. These results suggest that the rs405509 modulates the effect of e4/4 by controlling the level of APOE protein.

### INDUSTRIAL APPLICABILITY

The present invention not only provides APOE genotypes from genomic DNAs obtained from bloods or biological specimen, but also provides a method for more precisely predicting risk of dementia by analyzing genetic variations of re405509 in APOE promotor region. This invention may contribute to the development of diagnosis technologies in the future based on accumulated big data for dementia, together with clinical and pathological information, such as, MRI brain image, neuropsychological test, CSF test and amyloid-PET test, etc.

1. A method for diagnosing MIC (minor cognitive impairment) or Alzheimer's disease or predicting the risk of MIC or Alzheimer's disease by determining genetic variations, which indicate the MIC or AD, from a sample isolated from a subject and comprising nucleic acids, wherein the method comprises steps of,
(a) contacting the sample above with a reagent to confirm the presence or absence of the said genetic variations in genes encoding APOE E4 allele or genes selected from genetic products of the said genes encoding APOE E4 allele; and
(b) contacting the sample above with a reagent to detect the presence or absence of the said genetic variations in genes encoding rs405509 T allele of APOE promotor or genes selected from genetic products of the said genes encoding APOE E4 allele;
and wherein the presence of both genetic variations of APOE E4/E4 and rs405509 T/T indicates that the subject is a MIC or AD patient or indicates that the subject has a risk of MIC or AD disease.

2. The method of claim 1, wherein the method of determining the presence or absence of genetic variations of APOE E4/E4 and rs405509 T/T consists of constructing a complex of the said nucleic acids in single-stranded form and their complementary primer sets, and analyzing the presence or absence of the said genetic variations in the said complex.

3. The method of claim 2, wherein the determination of the presence or absence of genetic variations of APOE E4/E4 and rs405509 T/T is carried out by polymerase chain reaction, nuclease digestion, hybridization, southern blotting, restriction enzyme fragment polymorphism, sequencing, primer extension, single-stranded conformation polymorphism, or any combination of two or more of the above.

4. The method of claim 1, wherein the nucleic acids in the sample isolated from the subject are amplified.

5. The method of claim 1, wherein the nucleic acids in the sample isolated from the subject are genomic DNAs.

6. The method of claim 1, wherein the nucleic acids in the sample isolated from the subject are RNAs.

7. The method of claim 1, wherein the method further comprises obtaining images of cortical thickness, and wherein the cortical thickness atrophy determined by the images presents that the subject is a MIC or AD patient or presents that the subject has a risk of MIC or AD.

8. The method of claim 7, wherein the images of cortical thickness are MR brain images.

9. The method of any one of claims 1, 7 and 8, wherein the method further comprises performing any one or more tests selected from a group consisting of neuropsychological test, CSF test and amyloid-PET test.

10. A kit for diagnosing MIC or Alzheimer's disease or predicting the risk of MIC or Alzheimer's disease, which determines the presence or absence of APOE E4/E4 genetic variations in genes encoding APOE E4 allele or genes selected from the genetic products of the genes encoding APOE E4 allele; and further determines the presence or absence of rs405509 T/T genetic variations in genes encoding rs405509 T/T allele of APOE promotor or genes selected from the genetic products of the genes encoding rs405509 T/T allele of APOE promotor.

11. The kit of claim 10, wherein further comprises primer sets complementary to the APOE E4 allele and the APOE promotor rs405509 T allele, respectively, and enzymes for reaction.

12. The kit of claim 10, wherein the kit further comprises a manual for analyzing the genetic variations.

13. The kit of claim 10, wherein the kit further comprises a manual for amplifying nucleic acids.

14. The method of claim 10, wherein the kit further comprises a standard control.


SEQUENCE LISTING (6 sequences)

SEQ ID NO: 1 - DNA, 28 bp, Artificial Sequence
  Forward primer - APOE promoter
ggggtaccgaaagcagcggatccttgat

SEQ ID NO: 2 - DNA, 28 bp, Artificial Sequence
  Reverse primer - APOE promoter
cccctcgagcttcctgcctgtgattggc

SEQ ID NO: 3 - DNA, 28 bp, Artificial Sequence
  Forward primer - APOE rs405509 allele replacement
gaggagggtgtctggattactgggcgag

SEQ ID NO: 4 - DNA, 28 bp, Artificial Sequence
  Reverse primer - APOE rs405509 allele replacement
ctcgcccagtaatccagacaccctcctc

SEQ ID NO: 5 - DNA, 29 bp, Artificial Sequence
  Forward primer - APOE rs405509 allele replacement
gaggagggtgtctgtattactgggcgagg

SEQ ID NO: 6 - DNA, 29 bp, Artificial Sequence
  Reverse primer - APOE rs405509 allele replacement
cctcgcccagtaatacagacaccctcctc